(19) United States
(12) Patent Application Publication
(10) Pub. No.: US 2024/0139030 A1
(43) Pub. Date: May 2, 2024

(54) RAKAN GLAUCOMA DEVICE

(71) Applicant: Rakan Alhourani, Apple Valley, CA (US)

(72) Inventor: Rakan Alhourani, Apple Valley, CA (US)

(21) Appl. No.: 18/507,069

(22) Filed: Nov. 12, 2023

(51) Int. Cl. A61F 9/007

(52) U.S. Cl. CPC A61F 9/00781; A61F 2250/0013; A61F 2250/0097

(57) ABSTRACT

The present invention is a device that is used for glaucoma management by using an adjustable drainage device. And this device has an indicator to help patient checking the intraocular pressure hence seeking medical advice immediately as needed.

### CROSS-REFERENCES TO RELATED APPLICATIONS

Not Applicable

### STATEMENT REGARDING FEDERALLY SPONSORED RESEARCH OR DEVELOPMENT

Not Applicable

### FIELD OF THE INVENTION

The present invention generally relates to a device that drains aqueous humor from inside the eye in order to adjust intraocular pressure (TOP). In this device, the drainage flow rate and amount are adjustable and the IOP can be checked by the patient at any time.

### BACKGROUND OF THE INVENTION

Rakan glaucoma device has an application in managing glaucoma and checking the IOP directly by patient. This device can drain excessive intraocular fluid hence regulating and adjusting the IOP, as the drainage flow rate is self-adjusted depending on IOP and can be readjusted anytime. Also the existence of valve prevents back flow of aqueous humor and it prevents unwanted decrease in IOP (hypotony).

### SUMMARY OF THE INVENTION

The present invention is a drainage device that manages glaucoma using adjustable valves and shunts that precisely control aqueous humor outflow hence IOP. Also, this new device allows patients to self-check IOP.

### BRIEF DESCRIPTION OF THE SEVERAL VIEWS OF THE DRAWINGS

FIG. 1: General view of Rakan glaucoma device with sliding-seal piece blunt-sealing big portion of third part.

FIG. 2: General view of Rakan glaucoma device showing three parts separated with the sliding-seal piece moved apart for illustration.

FIG. 3: First part of Rakan glaucoma device.

FIG. 4: Separated components of the first part showing indicator-valve-seal piece apart from tube for illustration.

FIG. 5: Two-dimensional view of the first part showing the indicator-valve-seal piece in closed position.

FIG. 6: Two-dimensional view of the first part showing the indicator-valve-seal piece in open position.

FIG. 7: Two-dimensional view of the first part showing the indicator-valve-seal piece in open position with indicator window.

FIG. 8: First part of Rakan glaucoma device using a different-shape spring while the indicator-valve-seal piece in closed position.

FIG. 9: First part of Rakan glaucoma device using a different-shape spring while the indicator-valve-seal piece in open position.

FIG. 10: First part of Rakan glaucoma device using a different-shape spring with a spring-adjusting piece while the indicator-valve-seal piece in closed position.

FIG. 11: First part of Rakan glaucoma device using a contained-pressure container instead of spring.

FIG. 12: Two-dimensional view of the first part using a contained-pressure container instead of spring.

FIG. 13: Second part of Rakan glaucoma device with sliding-seal piece moved apart for illustration.

FIG. 14: Second part of Rakan glaucoma device using a cylindrical-shaped tube with sliding-seal piece moved apart for illustration.

FIG. 15: Second part of Rakan glaucoma device using a cylindrical-shaped tube with two tube-surface-valve openings and sliding-seal piece moved apart for illustration.

FIG. 16: Side view of second parts with two tube-surface-valve openings showing sliding-seal piece blunt-sealing the openings at different levels.

FIG. 17: Second part of Rakan glaucoma device using a cylindrical-shaped tube with five tube-surface-valve openings and sliding-seal piece moved apart for illustration.

FIG. 18: Side view of second part showing valves in an open position as valve-cover pieces are displaced away from tube-surface-valve openings and sliding-seal piece moved apart for illustration.

FIG. 19: Side view of second part showing valves in a closed position as tube-surface-valve openings are sealed with valve-cover pieces and sliding-seal piece moved apart for illustration.

FIG. 20: Different design of second part with spring connected to two valve-cover pieces from inside the tube.

FIG. 21: Side view of different designs of the second part.

FIG. 22: Above view of different designs of the second part.

FIG. 23: Different design of second part which does not involve a sliding-seal piece and has no connection to third part of Rakan glaucoma device.

FIG. 24: Third part of Rakan glaucoma device with sliding-seal piece moved apart for illustration.

FIG. 25: Third part using a cylindrical-shaped tube with different shapes and sizes of tube-surface openings with sliding-seal piece moved apart for illustration.

FIG. 26: General view of Rakan glaucoma device with sliding-seal piece blunt-sealing second and third parts completely.

FIG. 27: General view of Rakan glaucoma device with sliding-seal piece blunt-sealing portion of second part and all of third part.

FIG. 28: General view of Rakan glaucoma device with sliding-seal piece blunt-sealing small portion of third part.

### DETAILED DESCRIPTION OF THE INVENTION

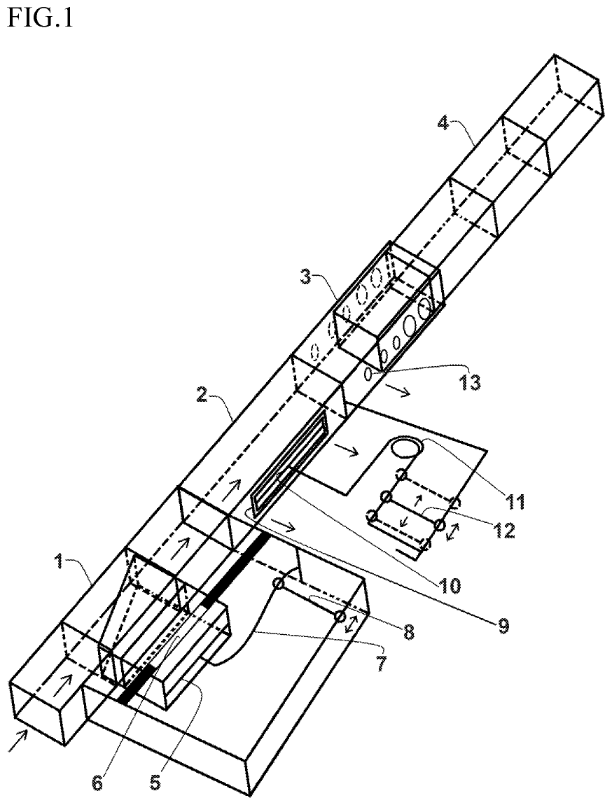

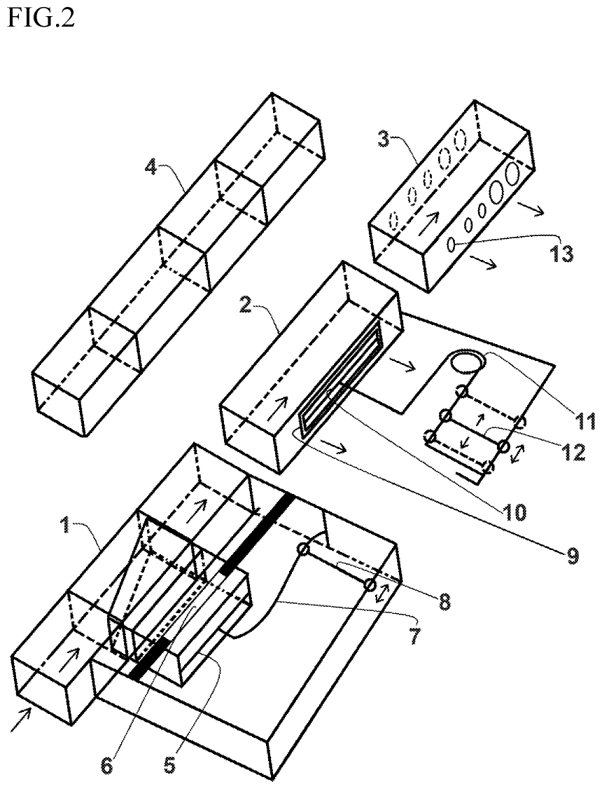

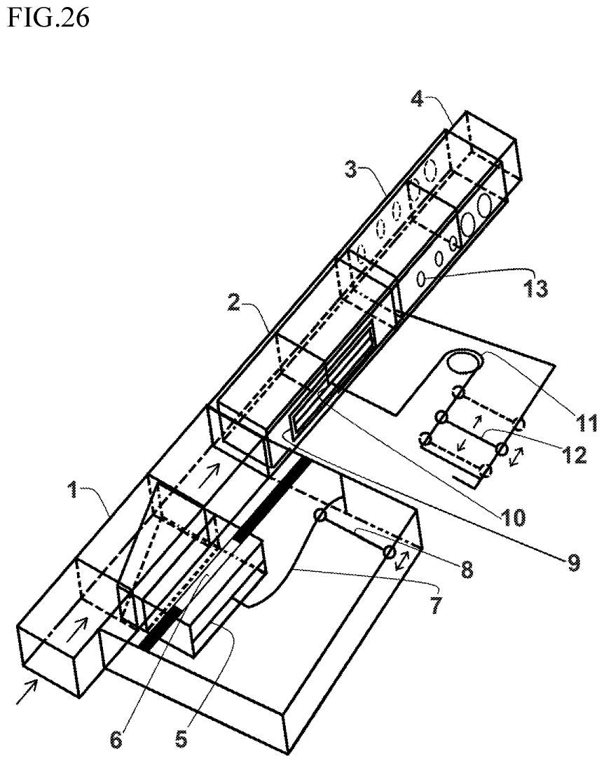

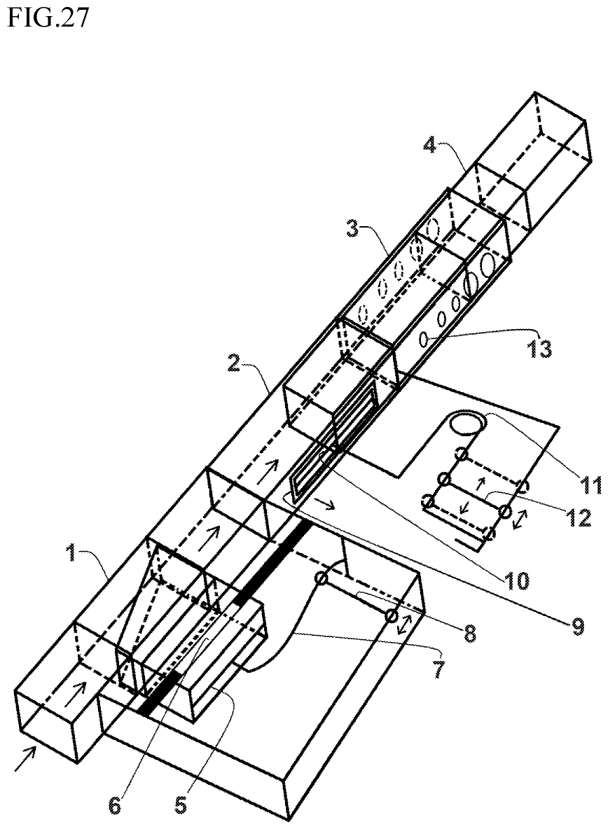

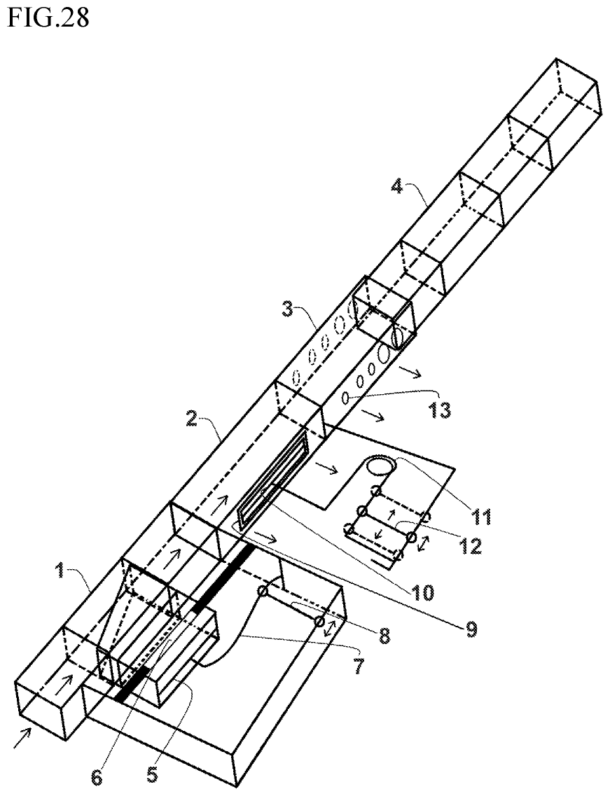

Rakan glaucoma device FIG. 1/FIG. 2/FIG. 26/FIG. 27/FIG. 28 consists of three parts.

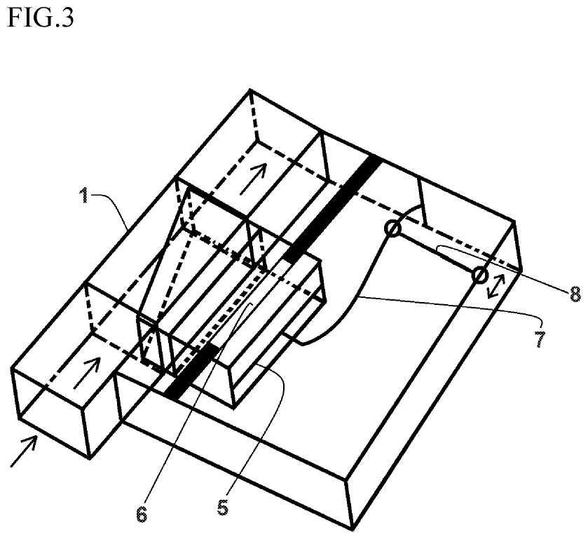

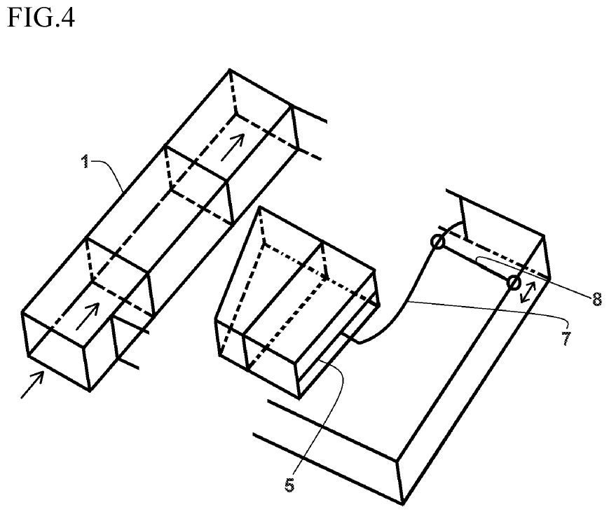

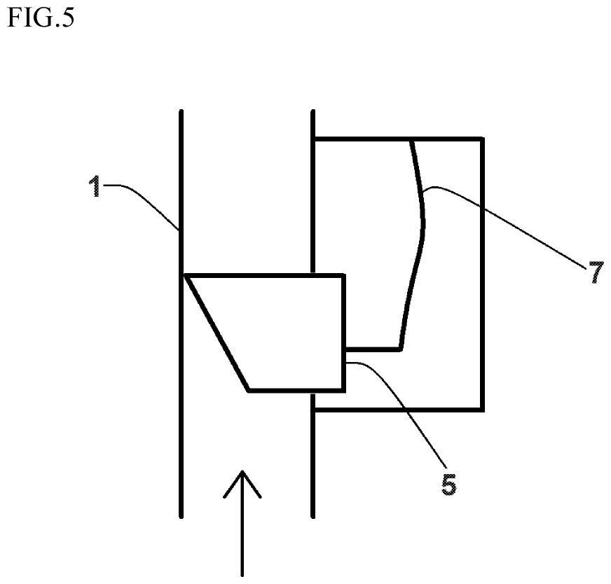

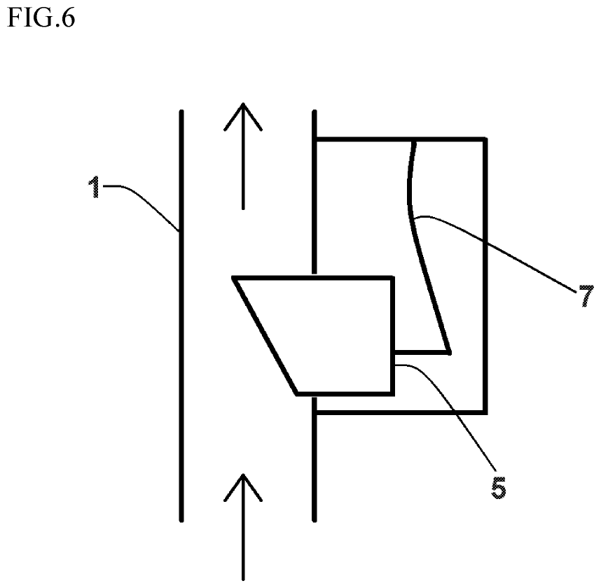

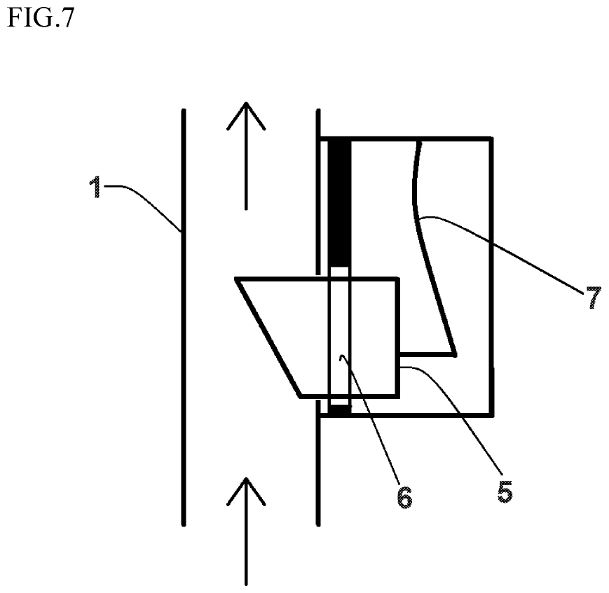

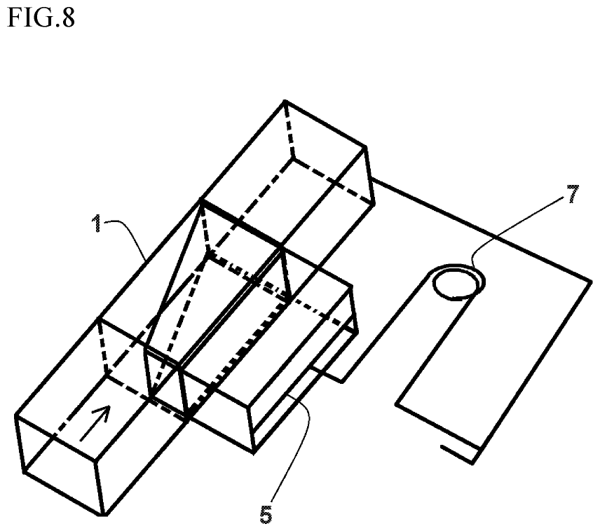

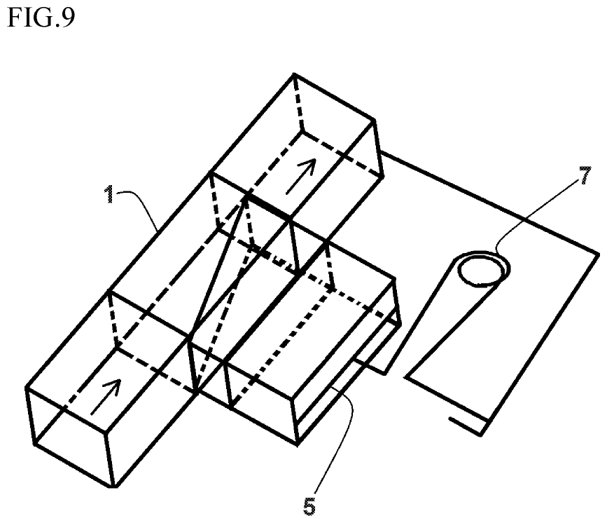

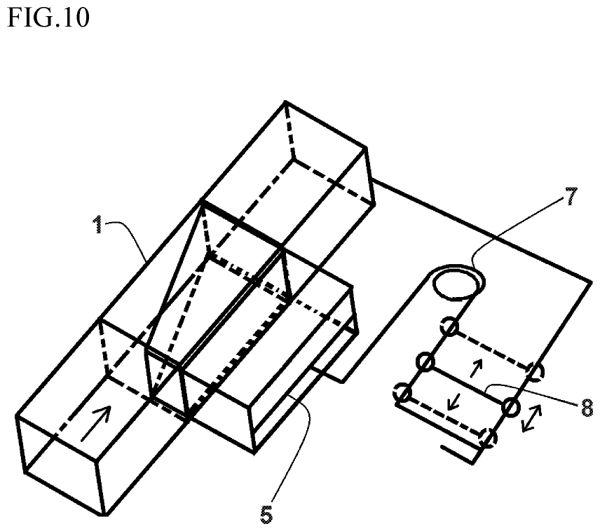

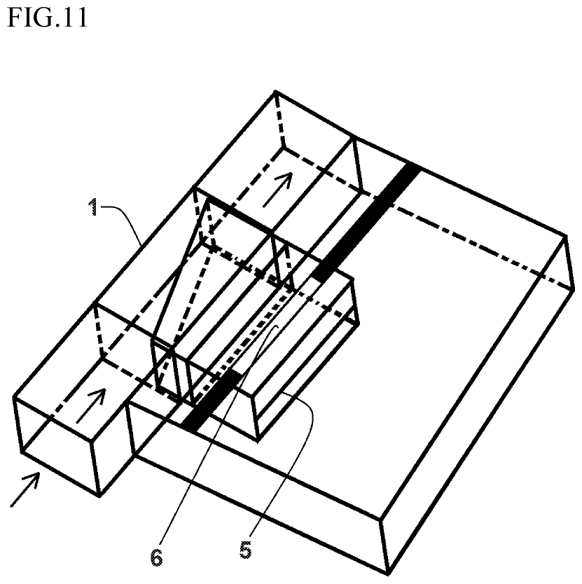

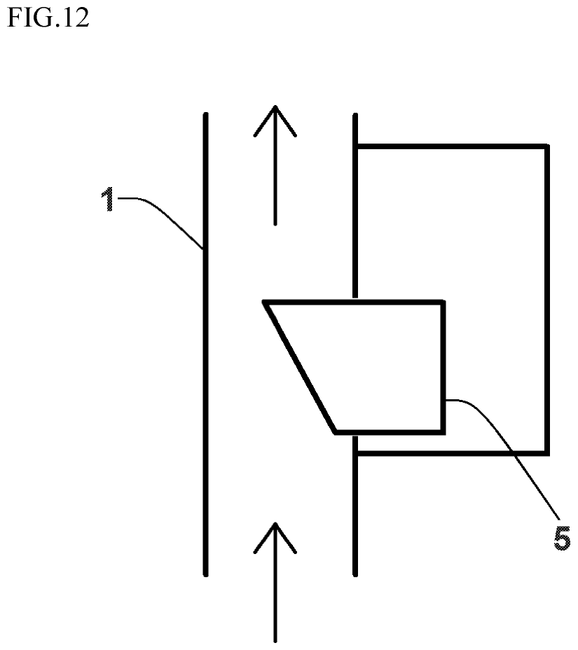

The first part FIG. 3/FIG. 4/FIG. 5/FIG. 6/FIG. 7/FIG. 8/FIG. 9/FIG. 10/FIG. 11/FIG. 12 comprises a tube 1 which could be in different shapes, that has a surface-opening which is configured to fit an indicator-valve-seal piece 5. At one side, the indicator-valve-seal piece 5 has a direct contact with fluid/aqueous humor inside the tube 1, with this fluid having a pressure hence generating force on the contacted side. At opposite/another side, there is a counteracting/opposing pressure/force generated by a contained-pressure container FIG. 11/FIG. 12 or by a spring structure 7 which could be in different shapes and that may have an adjustable piece 8 for adjusting power of the spring, as by moving the adjustable piece 8 in the directions shown by the illustrating-surrounding arrows, the length of arm will be adjusted hence adjusting the needed power to squeeze or release the spring 7.

The difference between the two pressures/forces—on the opposing sides
determines the direction and the amount of movement of the indicator-valve-seal piece 5, as if the fluid pressure/force is less than the counteracting pressure/force, then the indicator-valve-seal piece 5 will be in closed position by getting displaced towards the inside of the tube hence closing/sealing the tube and preventing flow of the fluid/aqueous humor as shown in FIG. 5/FIG. 8/FIG. 10 with the arrow inside the tube showing the direction of fluid flow. And if the fluid pressure/force is more than the counteracting pressure/force, then the indicator-valve-seal 5 piece will be in open position by getting displaced away from the inside of the tube hence opening the tube and allowing flow of the fluid as shown in FIG. 1/FIG. 2/FIG. 3/FIG. 6/FIG. 7/FIG. 9/FIG. 11/FIG. 12/FIG. 26/FIG. 27/FIG. 28 with arrows inside the tube showing the direction of the fluid flow.

The indicator-valve-seal piece 5 has a color and/or number scale that can be seen through an indicator window 6, and as the difference between the two pressures/forces is variable and depends mainly on the change of fluid pressure, the amount of displacement of the indicator-valve-seal piece 5 is variable and depends mainly on the change of fluid pressure, hence different color and/or number would be seen through the indicator window 6 that reflects fluid/aqueous humor pressure.

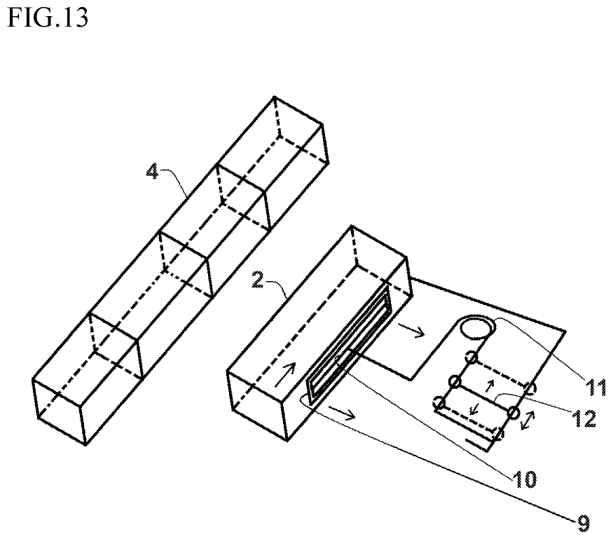

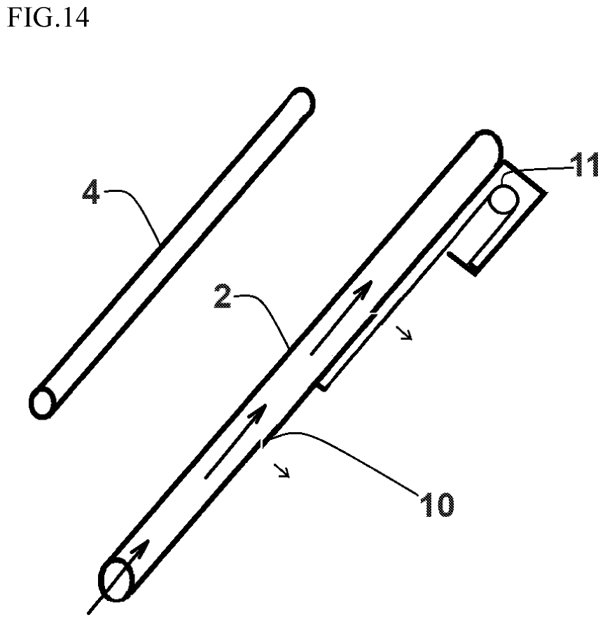

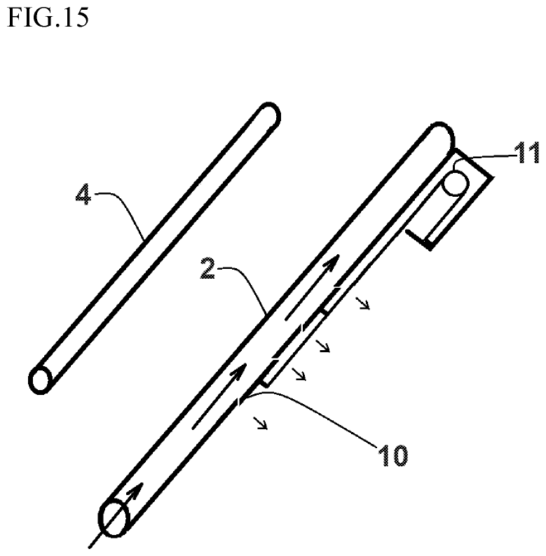

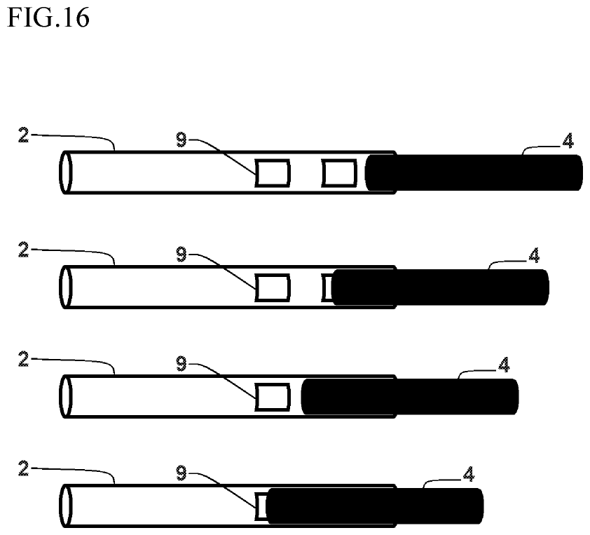

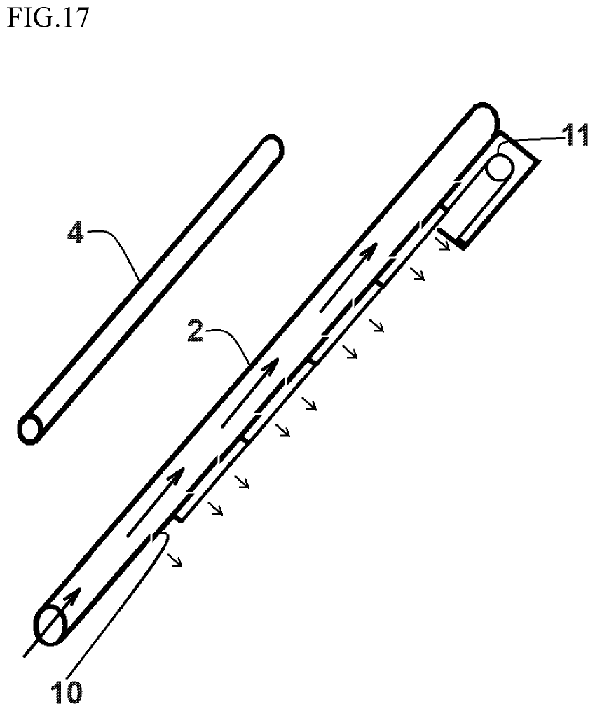

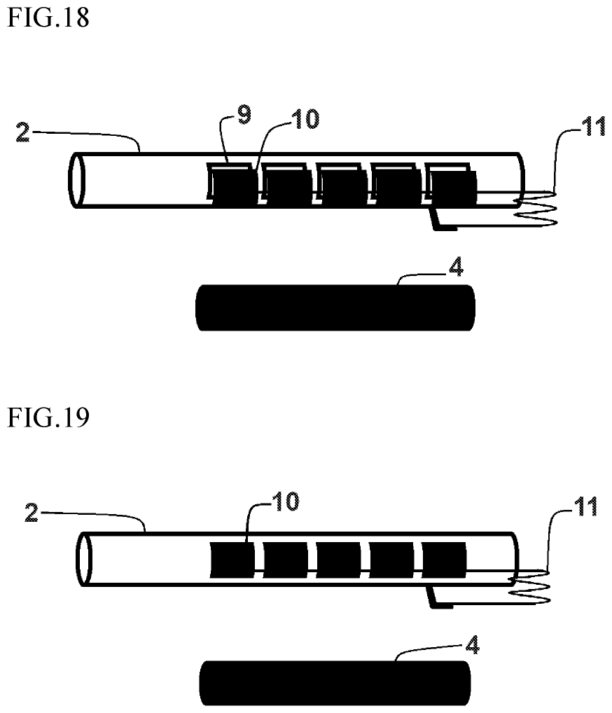

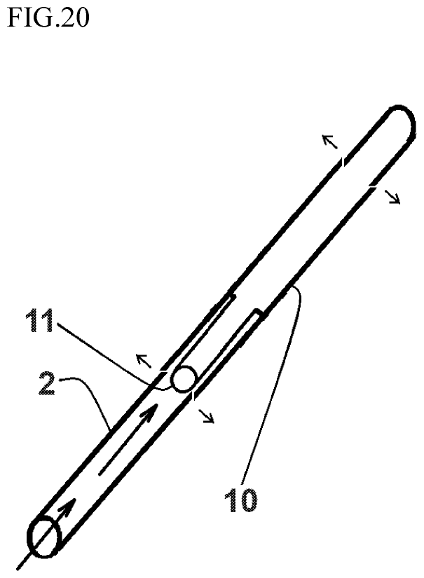

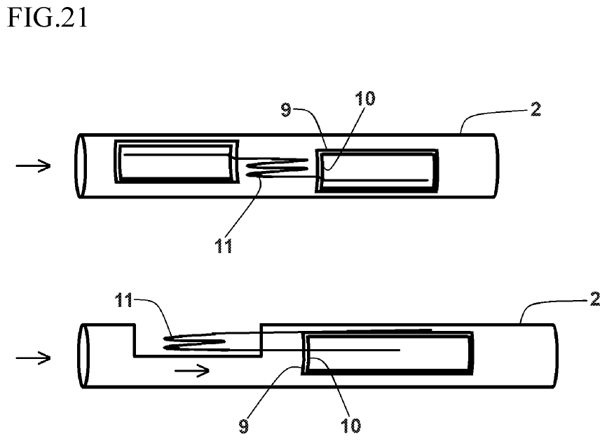

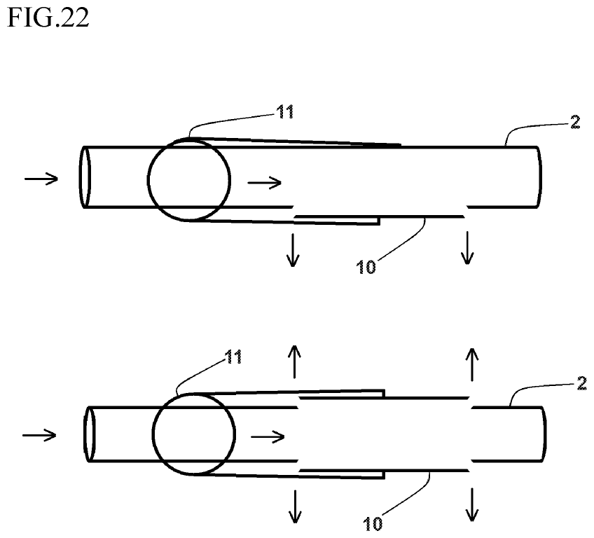

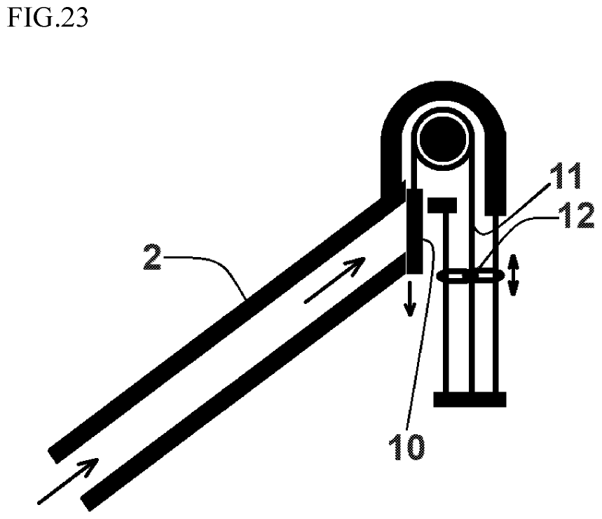

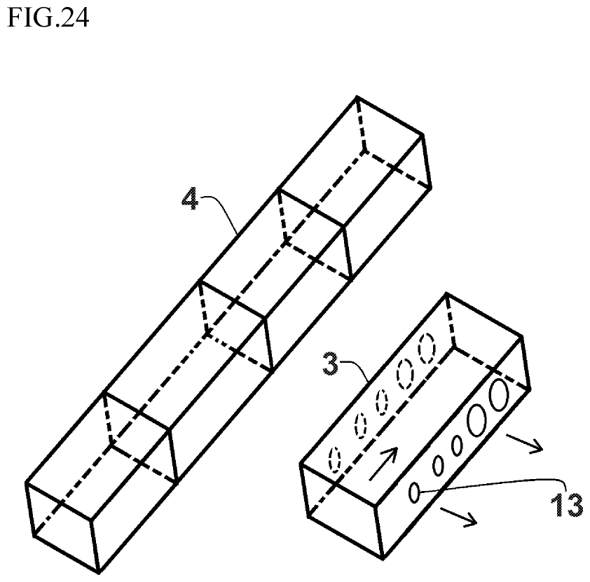

The second part FIG. 13/FIG. 14/FIG. 15/FIG. 16/FIG. 17/FIG. 18/FIG. 19/FIG. 20/FIG. 21/FIG. 22/FIG. 23 comprises a tube 2 which could be in different shapes and that has a tube-surface-valve opening 9 which is configured to be sealed by valve-cover piece 10. The second part 2 also comprises a sliding-seal piece 4, that is a blunt structure and it is configured to slide inside and blunt-seal the tube 2 lumen. The valve-cover piece is connected to a spring 11 that applies force on one side of the valve-cover piece 10 and pushes it against the tube-surface-valve opening 9 in order to seal it, while on the opposing side of the valve-cover piece 9 there is an opposing force that is generated by the pressure of the fluid/aqueous humor inside the tube 2. The difference between the two forces on the opposing sides determines the position of the valve-cover piece, as if the fluid pressure/force is less than the opposing force generated by spring 11, then the second part valve will be in closed position FIG. 19, as the tube-surface-valve opening 9 is sealed by valve-cover piece 10. And if the fluid pressure/force is more than the opposing force generated by spring 11, then the second part valve will be in open position FIG. 18, as the valve-cover piece 10 is displaced away from the tube-surface-valve opening 9 which allows fluid/aqueous humor drainage out of the tube hence decreasing intraocular pressure. The tube 2 could have one tube-surface-valve opening 9 and one valve-cover piece 10 as shown in FIG. 14 or more as shown in FIG. 15/FIG. 17 and the spring and openings could be in different designs and orientations FIG. 20/FIG. 21/FIG. 22.

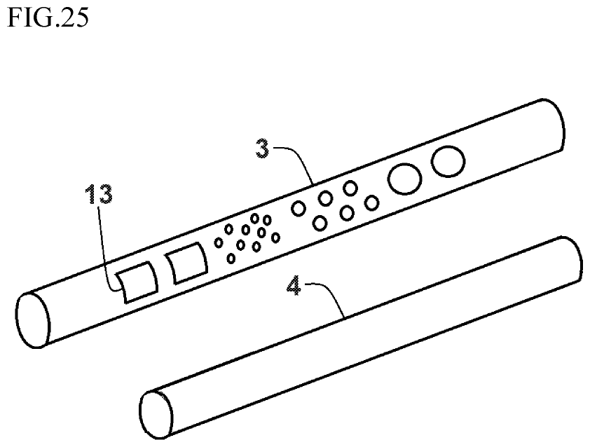

The force generated by the fluid/aqueous humor pressure depends on the size of surface area that is exposed to and in contact with the fluid, hence using the sliding-seal piece 4 to adjust the area of the valve-cover piece/pieces 10 that is exposed to fluid pressure FIG. 26/FIG. 27/FIG. 28 would adjust the generated force that opposes the force generated by the spring 11 as shown in FIG. 16 and by this we can adjust the amount of fluid/aqueous humor pressure needed to open valve and drain fluid out of the eye, hence adjusting intraocular pressure. Furthermore, the second part spring 11 could be in different shapes and it may have an adjustable piece 12 for adjusting power of the spring, as by moving the adjustable piece 12 in the directions shown by the illustrating-surrounding arrows, the length of arm will be adjusted hence adjusting the needed power to squeeze or release the spring 11. So, using the sliding-seal piece 4 and the adjustable piece 12 would allow us to precisely control the amount of fluid outflow/drainage and intraocular pressure. The sliding-seal piece 4 is used with both second and third part of Rakan glaucoma device, However the second part could be in a different design FIG. 23 without using sliding-seal piece 4 and without being connected to the third part. The third part FIG. 24/FIG. 25 comprises a tube 3 which could be in different shapes and that could have one surface-opening 13 or more that could be in different shapes which is configured to be sealed from inside the tube 3 by the sliding-seal piece 4. The sliding-seal piece could be adjusted to blunt seal all the surface-openings FIG. 26/FIG. 27 or to blunt-seal different numbers of the surface-openings FIG. 1/FIG. 28, and adjusting the number of the openings 13 sealed/unsealed determines the amount of aqueous humor/fluid drainage hence adjusting intraocular pressure.

1. An intraocular fluid drainage device comprises three parts: a first part comprising a tube, an indicator-valve-seal, an indicator window and a spring; said tube is connected to inside of the eye at one end and to a second part at other end; said tube has a surface opening; said surface opening configured to receive the indicator-valve-seal; said indicator-valve-seal is a movable blunt structure having colors and numbers indicator scale; said indicator scale configured to be seen through the indicator window; said indicator-valve-seal having direct contact with the fluid inside the tube at one side and with the spring at other side; said fluid inside the tube having a pressure reflecting intraocular pressure which generates force at contacted side of the indicator-valve-seal; said spring generating opposing force at other side of the indicator-valve-seal; said spring having an adjustable piece which adjusting the force needed to squeeze or release the spring;
Wherein direction and amount of movement of the indicator-valve-seal movement being determined by difference between the two forces generated by the fluid pressure and the spring;
Wherein if the fluid-pressure force is less than the spring force, the indicator-valve-seal would be displaced towards inside of the tube hence sealing the tube and preventing fluid flow and drainage;
Wherein if the fluid pressure force is more than the spring force, the indicator-valve-seal would be displaced away from inside of the tube hence opening the tube and allowing flow and drainage of fluid hence decreasing intraocular pressure;
wherein amount of opening is proportional to the fluid pressure force, hence increasing pressure would increase amount of opening hence increasing fluid flow and drainage;
wherein the amount of displacement of the indicator-valve-seal depending on the difference of the two opposing forces hence the color and number shown through the indicator window;
A second part comprising a tube, a sliding-seal, a valve-cover and a spring; said tube is connected to the first part at one end and to a third part at other end; said tube having a tube-surface-opening; said tube configured to receive the sliding-seal; said sliding-seal is a movable blunt structure configured to blunt-seal tubes of second and third parts from inside; said tube-surface-opening is configured to be sealed by the valve-cover; said valve-cover is movable and having direct contact with fluid inside the tube at one side and with the spring at other side; said fluid inside the tube having a pressure reflecting intraocular pressure which generating force at on side of the valve-cover; said spring generating opposing force at other side of valve-cover; said spring having an adjustable piece which adjusting the force needed to squeeze or release the spring; said force generated by fluid pressure depending on size of area of valve-cover exposed to fluid; said size of area of valve-cover exposed to fluid is determined by moving the sliding-seal;
Wherein the position of the valve-cover being determined by difference between the two forces generated by the fluid pressure and the spring;
Wherein if the fluid-pressure force is less than the spring force, the valve-cover would be in closed position as it would be displaced towards the tube-surface-opening hence sealing it and preventing fluid drainage;
Wherein if the fluid pressure force is more than the spring force, the valve-cover would be in open position as it would be displaced away from the tube-surface-opening hence opening it and allowing drainage of fluid hence decreasing intraocular pressure;
Wherein the force generated by fluid pressure is adjusted by adjusting the area of valve-cover exposed to fluid by moving the sliding-seal hence controlling the position of valve-cover and intraocular pressure;
A third part comprising a tube and a sliding-seal; said tube is connected to the second part at one end; said tube having surface-openings; said sliding-seal is a movable blunt structure configured to blunt-seal tube and surface-openings from inside;
Wherein number and portion of surface-openings sealed is controlled by moving the sliding-seal;
Wherein controlling the number of surface-openings and portion sealed controls the amount of fluid drainage hence controlling intraocular pressure.

2. the device of claim 1 wherein using force generated by a contained-pressure container as an opposing force to fluid pressure force at indicator-valve-seal at first part, instead of using a spring.
